(19) United States
(12) Patent Application Publication
(10) Pub. No.: US 2026/0245730 A1
(43) Pub. Date: Aug. 20, 2026

(54) AI-DRIVEN SYSTEM AND METHOD FOR PREDICTING THE READMISSION RISK OF A PATIENT FOLLOWING CARDIOVASCULAR OR CEREBROVASCULAR TREATMENT

(71) Applicant: SAIGEWARE INC, San Francisco, CA (US)

(72) Inventors: Prasad V R PANCHANGAM, Bengaluru (IN); Tejas A, Bengaluru (IN); Thejas B U, Bengaluru (IN)

(73) Assignee: SAIGEWARE INC, San Francisco, CA (US)

(21) Appl. No.: 19/054,633

(22) Filed: Feb. 14, 2025

(51) Int. Cl. G16H 50/30; G16H 50/70

(52) U.S. Cl. CPC G16H 50/30; G16H 50/70

(57) ABSTRACT

An AI-driven method predicts patient readmission risk after cardiovascular or cerebrovascular treatment, providing a percentage value for specific timeframes post-discharge. The method receives diverse patient data, including diagnoses, medications, vitals, lab reports, demographics, comorbidities, and admission/discharge information. This data is pre-processed into feature vectors, scaled, and then analyzed by an AI-engine employing multiple machine learning models. The AI-engine utilizes one-vs-one and one-vs-rest classifiers, a multi-class classifier, a binary classifier, and a meta-classifier to generate a comprehensive readmission risk prediction. This prediction, categorized by post-discharge timeframes, assists healthcare providers in tailoring interventions and monitoring strategies.

### TECHNICAL FIELD

The present disclosure relates to the field of predictive healthcare management. The present disclosure employs machine learning to predict the likelihood of patient readmission using Electronic Medical Record (EMR) data for patients admitted with cerebrovascular and cardiovascular conditions, facilitating proactive healthcare interventions.

### BACKGROUND

Hospital readmissions, particularly for patients with cardiovascular and cerebrovascular conditions, pose a persistent challenge to the healthcare system, leading to escalated costs and adverse impacts on patient well-being. Predicting readmission risk is crucial for proactive intervention and mitigation strategies. However, existing risk assessment methods often fall short. Traditional scoring systems like LACE and HOSPITAL, while utilizing factors such as length of stay, emergency history, lab results, and comorbidities, lack the granularity to capture the complex interplay of patient-specific risk factors. These generalized approaches, including manual or rule-based risk stratification, may overlook subtle patterns indicative of elevated readmission risk.

While machine learning (ML) offers promise in readmission risk prediction, current applications frequently fail to integrate essential patient-specific details. Omitting critical factors like comprehensive medication history, recent emergency department visits, and the full spectrum of comorbidities can hinder prediction accuracy and limit clinical utility. Moreover, many existing ML models focus solely on readmission within a fixed timeframe, neglecting the dynamic nature of patient recovery and the need for flexible prediction horizons to guide tailored care plans.

### SUMMARY

It is an object of the present disclosure to provide a system and method for predicting the readmission risk of a patient following cardiovascular or cerebrovascular treatment, while avoiding one or more drawbacks of prior art approaches. This object is achieved by the features of the independent claims. Further, implementation forms are apparent from the dependent claims, the description, and the figures.

According to a first aspect, an AI-driven system for predicting the readmission risk of a patient following a cardiovascular treatment, a cerebrovascular treatment, or both is provided. The readmission risk is represented by one or more percentage values, each of which associated with a temporal category, which is a timeframe after a patient discharge within which said patient is predicted to be readmitted. The system includes a memory unit for storing processing instructions and a processor for executing said processing instructions. Executing the processing instructions results in receiving input data comprising diagnoses data, medication data, vitals data, lab report data, demographic data, comorbidities data and admit-discharge data, preprocessing the input data resulting in the generation of feature vectors, scaling the feature vectors, transmitting the scaled feature vectors to an AI-engine employing multiple machine learning XGBoost models, and generating a readmission risk prediction for the patient. The multiple ML models comprise multiple one-vs-one classifier models, the number of which are a function of the number of the temporal categories, multiple one-vs-rest classifier models, the number of which are a function of the number of the temporal categories, a multi-class classifier model, a binary classifier, and a meta-learner.

According to a second aspect, an AI-driven method for predicting the readmission risk of a patient following a cardiovascular treatment, a cerebrovascular treatment, or both is provided. The readmission risk represented by one or more percentage values, each of which associated with a temporal category, which comprises a timeframe after a patient discharge within which said patient is predicted to readmit. The method includes receiving input data comprising diagnoses data, medication data, vitals data, lab report data, demographic data, comorbidities data, and admit-discharge data. The method includes preprocessing the input data resulting in the generation of feature vectors. The method includes scaling the feature vectors. The method includes transmitting the scaled feature vectors to an AI-engine that employs multiple ML models comprising multiple one-vs-one classifier models, the number of which are a function of the number of the temporal categories, multiple one-vs-rest classifier models, the number of which are a function of the number of the temporal categories, a multi-class classifier model, a binary classifier, and a meta-learner. The method includes generating a readmission risk prediction for the patient.

Preferably, the multiple temporal categories comprise a first category ranging between 1 to 7 days, a second category ranging between 7 to 30 days, a third category ranging between 30 to 60 days, a fourth category ranging between 60 to 120 days, a fifth category ranging between 120 to 180 days, a sixth category ranging between 180 to 365 days, and a seventh category exceeding 365 days.

Preferably, the lab data include lab test results pertaining to serum sodium, serum potassium, hemoglobin, blood glucose, blood urea nitrogen, and ECG.

Preferably, the vitals data include pulse rate, blood pressure, oxygen saturation, and respiratory rate.

Preferably, the medication data is categorized into RxNorm classes, each of which associated with a number, which is representative of the number of medications prescribed to the patient that pertain to the corresponding RxNorm class.

Preferably, the diagnoses data is categorized into organ system categories, each of which associated with a number, which is representative of the number of corresponding health conditions the patient is diagnosed with.

Preferably, the comorbidities data is categorized into Charlson Comorbidity Index (CCI) categories, each of which associated with a binary value, which is representative of said patient whether suffering from a comorbidity that pertains to the corresponding CCI category.

Preferably, the demographic data include patient race, age, height, weight, and gender.

Preferably, the binary classifier predicts if the patient gets readmitted within 120 days of discharge.

According to a third aspect, a computer program includes instructions for carrying out all the steps of the method, when said computer program is executed on a computer system.

These and other aspects of the present disclosure will be apparent from the implementation(s) described below.

### BRIEF DESCRIPTION OF FIGURES

Implementations of the present disclosure will now be described, by way of example only, with reference to the accompanying drawings, in which:

FIG. 1 is, according to an implementation of the present disclosure, a block diagram representing the system of present disclosure.

FIG. 2 is, according to an implementation of the present disclosure, a flowchart representing the method of the medication data preprocessor.

FIG. 3 is, according to an implementation of the present disclosure, a representation of the architecture of at least one XGBoost model.

FIG. 4 is, according to an implementation of the present disclosure, a flowchart representing the method of present disclosure.

FIG. 5 is an illustration of a computer system (e.g., an apparatus, a receiver, a converter, a replacer, and a sender) in which the various architectures and functionalities of the various previous implementations may be implemented.

### DETAILED DESCRIPTION

Implementations of the present disclosure provide an AI-driven system and method for predicting the readmission risk of a patient following cardiovascular and/or cerebrovascular treatment. To make solutions of the present disclosure more comprehensible for a person skilled in the art, the following implementations of the present disclosure are described with reference to the accompanying drawings.

Terms such as “a first,” “a second,” “a third,” and “a fourth” (if any) in the summary, claims, and foregoing accompanying drawings of the present disclosure are used to distinguish between similar objects and are not necessarily used to describe a specific sequence or order. It should be understood that the terms so used are interchangeable under appropriate circumstances, so that the implementations of the present disclosure described herein are, for example, capable of being implemented in sequences other than the sequences illustrated or described herein. Furthermore, the terms “include” and “have” and any variations thereof, are intended to cover a non-exclusive inclusion. For example, a process, a method, a system, a product, or a device that includes a series of steps or units, is not necessarily limited to expressly listed steps or units but may include other steps or units that are not expressly listed or that are inherent to such process, method, product, or device.

The present disclosure introduces an AI-driven system and method for predicting the readmission risk of a patient following cardiovascular and/or cerebrovascular treatment. This readmission risk is expressed as a probability, represented by one or more percentage values. Each percentage value is associated with a specific temporal category within a predefined set of temporal categories. In simpler terms, the purpose of the system and method of the present disclosure is to predict the number of days until the next readmission.

A temporal category defines a timeframe that commences after a patient's discharge. The system utilizes seven distinct temporal categories: a first category encompassing 1 to 7 days post-discharge, a second category ranging from 7 to 30 days, a third category spanning 30 to 60 days, a fourth category covering 60 to 120 days, a fifth category extending from 120 to 180 days, a sixth category encompassing 180 to 365 days, and a final seventh category for any readmission occurring beyond 365 days post-discharge. This multi-category approach allows for a more nuanced and informative prediction of readmission risk, enabling healthcare providers to tailor interventions and monitoring strategies based on the specific timeframes where a patient may be most vulnerable to readmission. In one implementation, the first to seventh categories are labelled from “class 0” to “class 6” respectively.

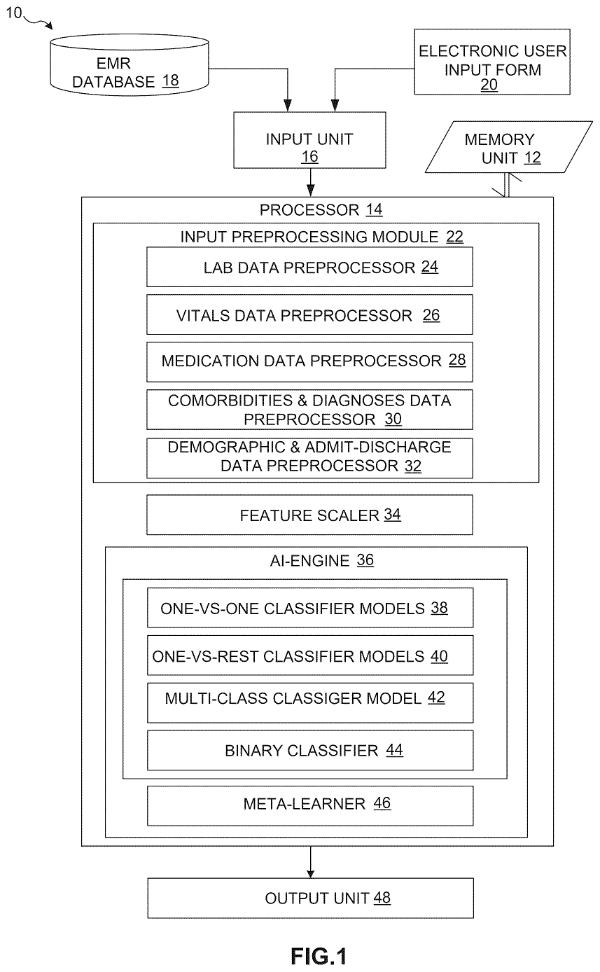

Referring to FIG. 1, the system 10 comprises a memory unit 12 for storing processing instructions and a processor 14 for executing said instructions to generate the readmission risk. The system 10 further includes an input unit 16 for receiving patient input data, which may include diagnoses data, medication data, comorbidities data, demographic data, vitals data, lab report data, and admit-discharge data. This input data can be queried from an Electronic Medical Records (EMR) database 18 hosted on a cloud server or an on-premises server at the hospital. In one implementation, the EMR database 18 itself is sourced from an Electronic Health Record (EHR) database.

Referring to FIG. 1, alternatively, the input data can be obtained through an electronic user input form 20, gathering information from the patient or their representative via a user interface facilitated by a dedicated application or website. In another implementation, the system 10 leverages a hybrid approach, sourcing data primarily from the EMR database 18 and supplementing any missing information through the user input form 20. This flexible data acquisition strategy ensures that the system 10 can operate effectively in diverse healthcare environments with varying levels of electronic record availability.

Referring to FIG. 1, the gathered input data is then uploaded to a cloud server responsible for housing, preprocessing, processing, and running inferences on the data to predict the risk of readmission. The cloud server (which is the processor 14) comprises an input preprocessing module 22, which includes a lab data preprocessor 24, a vitals data preprocessor 26, a medication data preprocessor 28, a comorbidities & diagnoses data preprocessor 30, and a demographic & admit-discharge data preprocessor 32. Each preprocessor within the input module 22 plays a crucial role in transforming the raw input data into the required feature vectors that will subsequently be used as input for an AI-engine 36. The specific functionalities of these preprocessors will be elaborated upon in the following body of text. This preprocessing stage ensures that the data is appropriately formatted, cleaned, and structured for optimal performance within the AI-engine 36, ultimately enhancing the accuracy and reliability of the readmission risk prediction.

Referring to FIG. 1, within the lab data preprocessor 24, various lab test results are analyzed, including serum sodium, serum potassium, haemoglobin, blood glucose, blood urea nitrogen, and ECG readings. Recognizing that the same lab tests may be conducted multiple times during a patient encounter, the system 10 utilizes both the mean and standard deviation of these results as features. This approach captures not only the average value but also the standard deviation in these measurements, providing a more comprehensive picture of the patient's physiological state. Furthermore, ECG results are encoded into categorical values based on the presence of “Atrial Fibrillation” or “Normal” findings, facilitating the integration of this crucial diagnostic information into the predictive model.

Referring to FIG. 1, in the vitals data preprocessor 26, key physiological parameters such as pulse rate, blood pressure, oxygen saturation, and respiratory rate are extracted and analyzed. To provide a more clinically relevant metric, blood pressure readings are converted to Mean Arterial Pressure (MAP). As with the rest, multiple vitals measurements may be recorded during a single encounter. Therefore, the system 10 is configured to calculate both the mean and standard deviation of these vitals to account for potential fluctuations and provide a more robust representation of the patient's physiological status over time. These derived features contribute valuable information to the overall assessment of readmission risk.

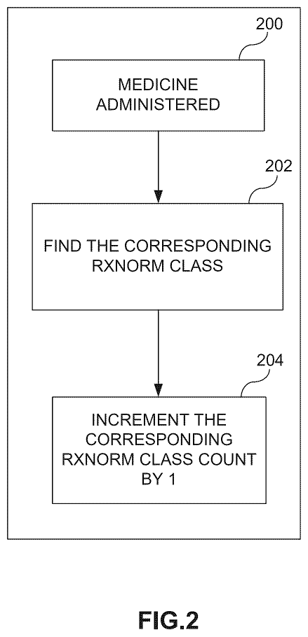

Referring to FIG. 1, in the medicine data preprocessor 28, the focus shifts to analyzing the various medications administered to the patient during their encounter. This analysis is performed using the algorithm illustrated in FIG. 2. Initially, a list of relevant RxNorm medicine classes is established, and the occurrence count for each class is set to zero. Then, for each medication administered to the patient (step 200), the corresponding RxNorm class is identified (step 202), and the occurrence count for that specific class is incremented by 1 (step 204). This process results in a set of features representing the frequency of each RxNorm medicine class administered during the encounter, providing valuable insights into the patient's treatment regimen and potential risk factors for readmission.

Referring to FIG. 1, the diagnosis data preprocessor 30 analyzes the ICD-10 diagnosis codes documented upon admission and the primary diagnosis code assigned to the patient. These diagnoses are then transformed into binary-valued features, each representing a specific organ system associated with the identified disease. For instance, a code like I25.2, which signifies a previous myocardial infarction, is interpreted as an indicator of a circulatory system issue. This approach facilitates a more structured and interpretable representation of the patient's diagnoses. Furthermore, comorbidities that contribute to the calculation of the Charlson Comorbidity Index (CCI) are also incorporated as binary-valued features. The CCI is a widely used tool for predicting mortality risk in patients with multiple comorbidities, and its inclusion enhances the system's ability to assess the overall health status and readmission risk of the patient.

Referring to FIG. 1, finally, the demographic & admit-discharge data preprocessor 32 handles information related to the patient's demographics and their admission and discharge circumstances. This includes data such as patient bed type, patient process type, admit-discharge settings, and demographic data. Patient bed types and process types are converted into binary or Boolean-valued features, indicating whether the patient utilized those specific services. Admit-discharge settings encompass data points like length of stay, number of emergency admissions in the previous six months, admit source, and discharge disposition. Admit source and discharge disposition are treated as binary variables, set to “1” if the patient was admitted from home and discharged to home, respectively. Demographic data includes patient race, age, height, weight, and gender. Race and gender are categorically encoded, while the numerical values of age, height, and weight are retained in their original form. This comprehensive processing of demographic and admit-discharge data provides valuable context for understanding the patient's individual circumstances and risk factors.

Referring to FIG. 1, following preprocessing, the resulting data, now referred to as “feature vectors” or simply “features” are passed through a feature scaler 34. Critically, these features are arranged in a predetermined order before being inputted to the feature scaler 34. This ensures consistency and proper alignment of the data for subsequent processing. The feature scaler 34 then applies a scaling function to standardize the range of values for each feature. This scaling process is essential for optimizing the performance of many machine learning algorithms, including the AI-engine 36 used in this system 10. The scaled features are then fed into the AI-engine 36 for readmission risk prediction.

In one implementation, prior to the feature scaling, in order to identify and retain the most significant features, statistical techniques such as analysis of variance (ANOVA) and chi-squared tests are employed. These tests were conducted to evaluate the relationship between the features and the readmission risk. Features that exhibited a p-value exceeding 0.05, signifying a weaker statistical relationship with the readmission risk, were eliminated.

A crucial step in assuring the accuracy and dependability of data for readmission risk analysis is to handle missing values. Sometimes, various features could have missing values, necessitating a systematic approach to address this issue. In one implementation, prior to the feature scaling, all features with missing values that accounted for less than 1% of the input data were selectively removed. Subsequently, a pooled regression method was applied to impute missing values for laboratory test results, vital signs, etc., following the removal of any outliers in the dataset.

Referring to FIG. 1, the AI-engine 36 employs a sophisticated ensemble approach, utilizing a total of 31 XGBoost models, each of which is a potent Gradient Boosted Decision Tree (GBDT) machine learning technique that is trained to capture different facets of the readmission risk prediction. Preferably, XGBoost Models of version 1.7.6 are employed.

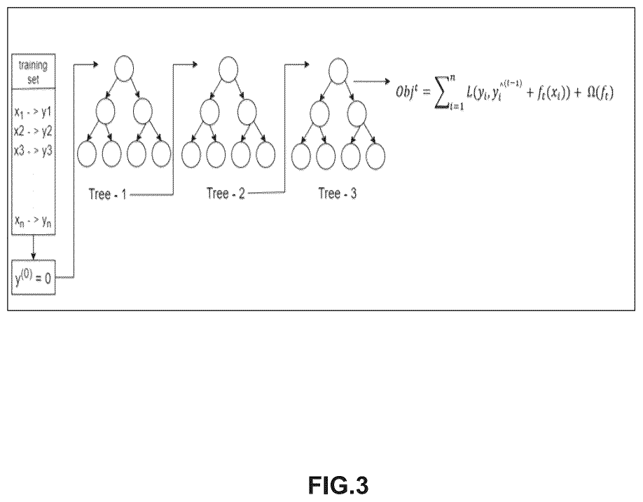

In one implementation, at least one XGBoost model, the architecture of each of which represented by FIG. 3, is configured to minimize the loss L^{(t)} as represented by the following equation #1:

$${L}^{( t )} = {\sum }_{i = 1}^{n} l ( {y}_{i} , {\hat{y}}_{i}^{( t - 1 )} + {f}_{t} ( {x}_{i} ) ) + Ω ( {f}_{t} )$$

Now, Ω(fₜ) is represented by the following equation #2:

$$Ω ( {f}_{t} ) = γ T + \frac{1}{2 λ {\vert w \vert}^{2}} = γ T + 1 / 2 λ {\sum }_{j = 1}^{T} {w}_{j}^{2}$$

where fₜ represents the set of base learners. Ω is the regularization function. xᵢ is the ith input to the model among n training points. yᵢ is actual label of the ith input, and

$${\hat{y}}_{i}^{( t - 1 )}$$

is the output predicted by the (t−1) base learner.

Consider the following equation #3:

$${g}_{i} = \frac{\partial L ( {y}_{i} , {\hat{y}}_{i}^{( t - 1 )} )}{\partial {\hat{y}}_{i}^{( t - 1 )}}$$

Consider the following equation #4:

$${h}_{i} = \frac{{\partial }^{2} L ( {y}_{i} , {\hat{y}}_{i}^{( t - 1 )} )}{\partial {\hat{y}}_{i}^{{( t - 1 )}^{2}}}$$

Now, by using Taylor series expansion and expanding up to 3 terms, equation #1 can be re-written with the help of equations #2, 3 and 4 as the following equation #5:

$${L}^{t} = {\sum }_{j = 1}^{T} [ ( \sum {g}_{i} ) {w}_{j} + 1 / 2 ( \sum {h}_{i} + λ ) {\sum }_{j = 1}^{T} {w}_{j}^{2} ] + γ T$$

Let ‘Gⱼ=Σgᵢ’ and ‘Hⱼ=Σhᵢ’ whereby equation #5 changes to the following equation #6:

$${L}^{( t )} = {\sum }_{j = 1}^{T} [ {G}_{j} {w}_{j} + \frac{1}{2} ( {H}_{j} + λ ) {w}_{j}^{2} ] + rT$$

Solve equation #6 and find the best

$$‘ {w}_{j}^{*} ’$$

represented by equation #7:

$${w}_{j}^{*} = - \frac{{G}_{j}}{{H}_{j} + λ}$$

The following equation #8 provides the total loss computed while training the XGBoost model:

$$L = - \frac{1}{2} {\sum }_{j = 1}^{T} \frac{{G}_{j}}{{H}_{j} + λ} + rT$$

In this implementation, hyperparameter tuning was used to optimize the model's performance and make it more accurate. The selected hyperparameters included the maximum depth of the decision tree (max_depth), subsample ratio (subsample), regularization lambda (reg_lambda), regularization alpha (reg_alpha), minimum child weight (min_child_weight), gamma, column subsampling at the tree level (colsample_by_tree), column subsampling at the level (colsample_by_level), and the maximum step size for updates (max_delta_step). The hyperparameters were selected using Grid Search and Bayesian Optimization. The parameters were selected based on their performance on the validation data.

In one implementation, at least one XGBoost model was trained in a distributed environment of 40 CPUs with 256 GB RAM. The XGBoost library implementation in Python was used for training the model. The objective function of the model was set to “multi: softmax”.

Referring to FIG. 1, specifically, 21 of these XGBoost models are one-vs-one classifier models 38. This number, 21, arises from the number of possible pairwise combinations of the seven temporal categories, calculated as 7!/(2!*(7−2)!)=21. Each one-vs-one model 38 is a binary classification model that focuses on discriminating between a specific pair of temporal categories. This approach allows the system to make nuanced predictions about the most likely timeframe for readmission if it occurs.

Referring to FIG. 1, in addition to the one-vs-one models 38, the AI-engine 36 includes seven one-vs-rest binary classifier models 40. Each of these one-vs-rest models 40 is trained to predict whether a patient belongs to a particular temporal category compared to all other categories combined. This provides a complementary perspective to the pairwise comparisons, further enhances the system's 10 predictive capabilities.

Referring to FIG. 1, furthermore, a multi-class classifier 42 is employed to directly predict the specific temporal category to which a patient belongs, providing a single, unified prediction.

Finally, a binary classifier 44 is trained to predict whether the patient will be readmitted within 120 days of discharge, encompassing the first four temporal categories. This specific prediction offers critical information for short-term monitoring and intervention strategies. This ensemble approach, combining multiple XGBoost models with diverse training objectives, contributes to the robustness and accuracy of the readmission risk prediction.

Referring to FIG. 1, to further refine the prediction, the outputs of these individual models 38, 40, 42 and 44 are then stacked and used as input to a meta-learner 46. The meta-learner 46 is trained on the collective wisdom of the ensemble, learning to weigh and combine the predictions of the individual models 38, 40, 42 and 44 optimally. The meta-learner 46 itself is a multi-class classifier model that produces the final readmission risk prediction. This prediction is then passed to an output unit 48, which is essentially a software application designed to transform the raw output of the meta-learner 46 into a human-readable format. This ensures that the prediction is presented in a clear, understandable manner, facilitating its effective use by healthcare providers.

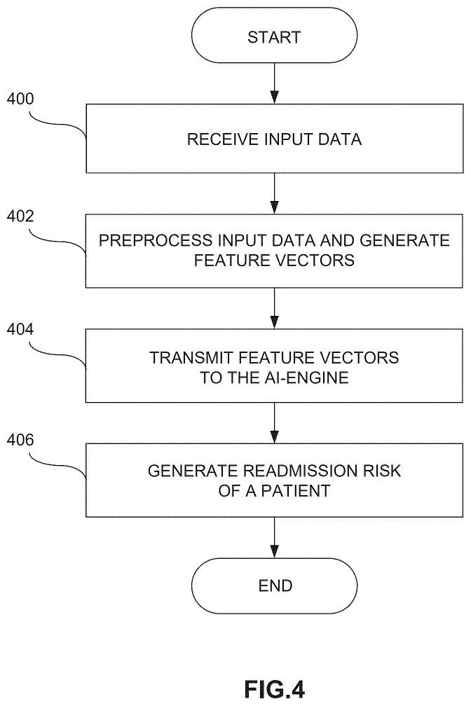

Referring to FIG. 4, the method for predicting the readmission risk of a patient following cardiovascular and/or cerebrovascular treatment begins with receiving (step 400) patient input data. This data encompasses a variety of relevant information, including diagnoses data, medication data, comorbidities data, demographic data, vitals data, lab report data, and admit-discharge data. The input data can be obtained from a variety of sources, including an Electronic Medical Records (EMR) database, an electronic user input form, or a combination of both.

Referring to FIG. 4, once the input data is gathered, it is uploaded to a cloud server for preprocessing (step 402). The cloud server utilizes an input preprocessing module, which includes several specialized preprocessors designed to handle different types of data. The lab data preprocessor analyzes lab test results, such as serum sodium, serum potassium, haemoglobin, blood glucose, blood urea nitrogen, and ECG readings. It utilizes both the mean and standard deviation of these results to capture a comprehensive picture of the patient's physiological state. The vitals data preprocessor extracts and analyzes key physiological parameters like pulse rate, blood pressure, oxygen saturation, and respiratory rate. Blood pressure readings are converted to Mean Arterial Pressure (MAP), and both the mean and standard deviation of these vitals are calculated to account for potential fluctuations.

Referring to FIG. 4, the medicine data preprocessor analyzes the medications administered to the patient using the algorithm illustrated in FIG. 2. This algorithm involves identifying the RxNorm class for each medication and incrementing the occurrence count for that class, resulting in a set of features representing the frequency of each RxNorm medicine class administered. The diagnosis data preprocessor analyzes ICD-10 diagnosis codes and transforms them into binary-valued features representing specific organ systems. It also incorporates comorbidities that contribute to the calculation of the Charlson Comorbidity Index (CCI). Finally, the demographic & admit-discharge data preprocessor handles information related to the patient's demographics, admission circumstances, and discharge circumstances, including data such as patient bed type, patient process type, admit-discharge settings, and demographic data.

Referring to FIG. 4, after preprocessing, the resulting features are arranged in a predetermined order and passed through a feature scaler to standardize the range of values. The scaled features are then transmitted (step 404) to the AI-engine for readmission risk prediction. The AI-engine employs an ensemble approach, utilizing 31 XGBoost models. These include 21 one-vs-one classifier models, each discriminating between a specific pair of temporal categories; seven one-vs-rest binary classifier models, each predicting whether a patient belongs to a particular temporal category compared to all others; a multi-class classifier to directly predict the specific temporal category; and a binary classifier to predict readmission within 120 days.

Referring to FIG. 4, the outputs of these individual models are then stacked and used as input to a meta-learner, which is a multi-class classifier model that generated (step 406) the final readmission risk prediction. This prediction is then passed to an output unit, which transforms the raw output into a human-readable format for use by healthcare providers.

In conclusion, the present disclosure details a novel system and method for predicting the readmission risk of patients following cardiovascular and/or cerebrovascular treatment. By leveraging a sophisticated AI-engine and a comprehensive approach to data acquisition and preprocessing, the system provides a nuanced and informative prediction of readmission risk across multiple temporal categories. This enables healthcare providers to tailor interventions and monitoring strategies to individual patient needs, potentially leading to improved patient outcomes and reduced healthcare costs associated with readmissions. The system's flexibility and adaptability to different healthcare data environments further enhance its practical utility and potential for widespread adoption.

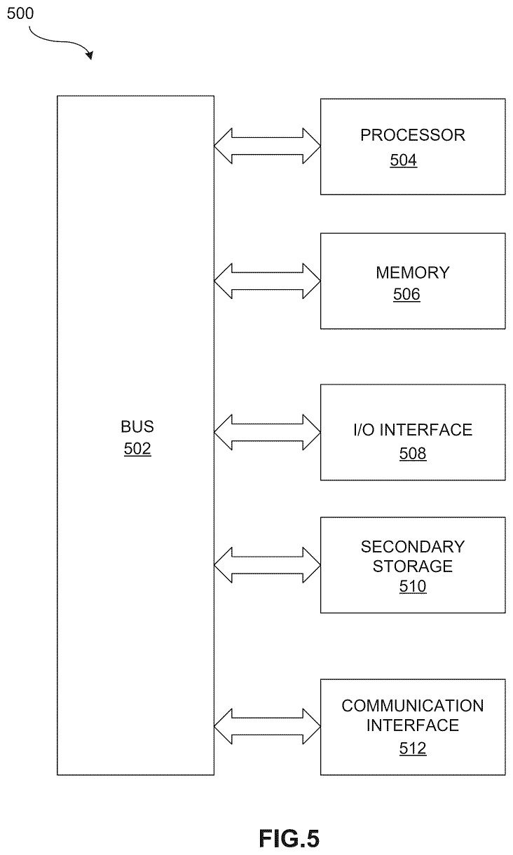

FIG. 5 is an illustration of a computer system (e.g., an apparatus) in which the various architectures and functionalities of the various previous implementations may be implemented. As shown, the computer system 500 includes at least one processor 504 that is connected to a bus 502, wherein the computer system 500 may be implemented using any suitable protocol, such as Peripheral Component Interconnect, PCI-Express, Accelerated Graphics Port, AGP, Hyper Transport, or any other bus or point-to-point communication protocol. The computer system 500 also includes a memory 506.

Control logic (software) and data are stored in the memory 506 which may take a form of random-access memory, RAM. In the present disclosure, a single semiconductor platform may refer to a sole unitary semiconductor-based integrated circuit or chip. It should be noted that the term single semiconductor platform may also refer to multi-chip modules with increased connectivity which simulate on-chip modules with increased connectivity which simulate on-chip operation, and make substantial improvements over utilizing a conventional central processing unit, CPU, and bus implementation. Of course, the various modules may also be situated separately or in various combinations of semiconductor platforms per the desires of the user.

The computer system 500 may also include a secondary storage 510. The secondary storage 510 includes, for example, a hard disk drive and a removable storage drive, representing a floppy disk drive, a magnetic tape drive, a compact disk drive, a digital versatile disk, a DVD, drive, a recording device, universal serial bus, USB, flash memory. The removable storage drives at least one of reads from and writes to a removable storage unit in a well-known manner. Computer programs, or computer control logic algorithms, may be stored in at least one of the memory 506 and the secondary storage 510. Such computer programs, when executed, enable the computer system 500 to perform various functions as described in the foregoing. The memory 506, the secondary storage 510, and any other storage are possible examples of computer-readable media.

In an implementation, the architectures and functionalities depicted in the various previous figures may be implemented in the context of the processor 504, a graphics processor coupled to a communication interface 512, an integrated circuit (not shown) that is capable of at least a portion of the capabilities of both the processor 504 and a graphics processor, a chipset (namely, a group of integrated circuits designed to work and sold as a unit for performing related functions, and so forth).

Furthermore, the architectures and functionalities depicted in the various previous-described figures may be implemented in a context of a general computer system, a circuit board system, a game console system dedicated to entertainment purposes, or an application-specific system. For example, the computer system 500 may take the form of a desktop computer, a laptop computer, a server, a workstation, a game console, or an embedded system.

Furthermore, the computer system 500 may take the form of various other devices including, but not limited to a personal digital assistant, PDA, device, a mobile phone device, smartphone, a television, and so forth. Additionally, although not shown, the computer system 500 may be coupled to a network (for example, a telecommunications network, a local area network, LAN, a wireless network, a wide area network, WAN, such as the Internet, a peer-to-peer network, a cable network, or the like) for communication purposes through an input/output, I/O, interface 508.

It should be understood that the arrangement of components illustrated in the figures described is exemplary and that other arrangements may be possible. It should also be understood that the various system components (and means) defined by the claims, described below, and illustrated in the various block diagrams represent components in some systems configured according to the subject matter disclosed herein. For example, one or more of these system components (and means) may be realized, in whole or in part, by at least some of the components illustrated in the arrangements illustrated in the described figures.

In addition, while at least one of these components is implemented at least partially as an electronic hardware component, and therefore constitutes a machine, the other components may be implemented in software that when included in an execution environment constitutes a machine, hardware, or a combination of software and hardware.

Although the present disclosure and its advantages have been described in detail, it should be understood that various changes, substitutions, and alterations can be made herein without departing from the spirit and scope of the present disclosure as defined by the appended claims.

What is claimed is:

1. An AI-driven system for predicting the readmission risk of a patient following a cardiovascular treatment, a cerebrovascular treatment, or both, said readmission risk represented by one or more percentage values, each of which associated with a temporal category, which comprises a timeframe after a patient discharge within which said patient is predicted to readmit, said system comprising:
(a) a memory unit storing processing instructions;
(b) a processor configured to execute said processing instructions to:
(i) receive input data comprising diagnoses data, medication data, vitals data, lab report data, demographic data, comorbidities data, and admit-discharge data;
(ii) preprocess said input data resulting in the generation of feature vectors;
(iii) scale said feature vectors;
(iv) transmit said scaled feature vectors to an AI-engine employing multiple machine learning XGBoost models comprising:
a. multiple one-vs-one classifier models, the number of which are a function of the number of said temporal categories;
b. multiple one-vs-rest classifier models, the number of which are a function of the number of said temporal categories;
c. a multi-class classifier model;
d. a binary classifier; and
e. a meta-learner; and
(v) generate a readmission risk prediction for said patient.

2. Said system of claim 1, wherein said multiple temporal categories comprise a first category ranging between 1 to 7 days, a second category ranging between 7 to 30 days, a third category ranging between 30 to 60 days, a fourth category ranging between 60 to 120 days, a fifth category ranging between 120 to 180 days, a sixth category ranging between 180 to 365 days, and a seventh category exceeding 365 days.

3. Said system of claim 1, wherein said lab data include lab test results pertaining to serum sodium, serum potassium, hemoglobin, blood glucose, blood urea nitrogen, and ECG.

4. Said system of claim 1, wherein said vitals data include pulse rate, blood pressure, oxygen saturation, and respiratory rate.

5. Said system of claim 1, wherein said medication data is categorized into RxNorm classes, each of which associated with a number, which is representative of the number of medications prescribed to said patient that pertain to the corresponding RxNorm class.

6. Said system of claim 1, wherein said diagnoses data is categorized into organ system categories, each of which associated with a number, which is representative of the number of corresponding health conditions said patient is diagnosed with.

7. Said system of claim 1, wherein said comorbidities data is categorized into Charlson Comorbidity Index (CCI) categories, each of which associated with a binary value, which is representative of said patient whether suffering from a comorbidity that pertains to the corresponding CCI category.

8. Said system of claim 1, wherein said admit-discharge data include patient bed type, patient process type, length of stay, number of emergency-admits in the previous six months, admit source, and discharge disposition.

9. Said system of claim 1, wherein said demographic data include patient race, age, height, weight, and gender.

10. Said system of claim 1, wherein said binary classifier predicts if said patient gets readmitted within 120 days of discharge.

11. An AI-driven method for predicting the readmission risk of a patient following a cardiovascular treatment, a cerebrovascular treatment, or both, said readmission risk represented by one or more percentage values, each of which associated with a temporal category, which comprises a timeframe after a patient discharge within which said patient is predicted to readmit, said method comprising:
(a) receiving input data comprising diagnoses data, medication data, vitals data, lab report data, demographic data, comorbidities data, and admit-discharge data;
(b) preprocessing said input data resulting in the generation of feature vectors;
(c) scaling said feature vectors;
(d) transmitting said scaled feature vectors to an AI-engine that employs multiple ML models comprising multiple one-vs-one classifier models, the number of which are a function of the number of said temporal categories, multiple one-vs-rest classifier models, the number of which are a function of the number of said temporal categories, a multi-class classifier model, a binary classifier, and a meta-learner; and
(e) generating a readmission risk prediction for said patient.

12. Said method of claim 11, wherein said multiple temporal categories comprise a first category ranging between 1 to 7 days, a second category ranging between 7 to 30 days, a third category ranging between 30 to 60 days, a fourth category ranging between 60 to 120 days, a fifth category ranging between 120 to 180 days, a sixth category ranging between 180 to 365 days, and a seventh category exceeding 365 days.

13. Said method of claim 11, wherein said lab data include lab test results pertaining to serum sodium, serum potassium, hemoglobin, blood glucose, blood urea nitrogen, and ECG.

14. Said method of claim 11, wherein said vitals data include pulse rate, blood pressure, oxygen saturation, and respiratory rate.

15. Said method of claim 11, wherein said medication data is categorized into RxNorm classes, each of which associated with a number, which is representative of the number of medications prescribed to said patient that pertain to the corresponding RxNorm class.

16. Said method of claim 11, wherein said diagnoses data is categorized into organ system categories, each of which associated with a number, which is representative of the number of corresponding health conditions said patient is diagnosed with.

17. Said method of claim 11, wherein said comorbidities data is categorized into Charlson Comorbidity Index (CCI) categories, each of which associated with a binary value, which is representative of said patient whether or not suffering from a comorbidity that pertains to the corresponding CCI category.

18. Said method of claim 11, wherein said admit-discharge data include patient bed type, patient process type, length of stay, number of emergency-admits in the previous six months, admit source, and discharge disposition.

19. Said method of claim 11, wherein said binary classifier predicts if said patient gets readmitted within 120 days of discharge.

20. A computer-readable medium containing program instructions executable by a processor to perform the method steps of claim 11.
